(19) United States
(12) Patent Application Publication
(10) Pub. No.: US 2025/0381141 A1
(43) Pub. Date: Dec. 18, 2025

(54) Formulation of lecithin-modified calcium phosphate nanoparticles with an enhanced cellar uptake as a carrier for bisphosphonates and a method of preparing thereof

(71) Applicant: POLITECHNIKA WARSZAWSKA, Warszawa (PL)

(72) Inventors: Michal WOJASINSKI, Warszawa (PL); Jolanta CZERSKA-DUSZAK, Warszawa (PL); Joanna LATOCHA, Warszawa (PL); Artur MALOLEPSZY, Warszawa (PL); Tomasz CIACH, Warszawa (PL)

(21) Appl. No.: 18/867,537

(22) PCT Filed: May 24, 2023
(86) PCT No.: PCT/IB2023/055336

§ 371 Date: Nov. 20, 2024

(30) Foreign Application Priority Data
May 24, 2022 (PL) P.441261

(51) Int. Cl. A61K 9/16; A61K 31/663; A61K 31/675; B82Y 5/00

(52) U.S. Cl. CPC A61K 9/1611; A61K 9/1617; A61K 9/1694; A61K 31/663; A61K 31/675; B82Y 5/00

(57) ABSTRACT

Formulation of nanoparticles of calcium phosphate, preferably hydroxyapatite, said nanoparticles being modified with lecithin, preferably phosphatidylcholine, said formulation having an enhanced cellular uptake and being a carrier for bisphosphonate, characterised in that bisphosphonate is selected from the group of bisphosphonate drugs approved for medical use, the group comprising alendronate and zoledronate, bisphosphonate is encapsulated in calcium phosphate nanoparticles in an amount up to 40% by mass, and the nanoparticles are less than 200 mn in size. Method of obtaining said formulation, comprising the steps: a. dissolving Ca(N03)2. 4H2O in a lecithin solution, b. dissolving (NH4)2HP04 in a bisphosphonate solution, c. adjusting the pH of the solution resulting from step a. and of the solution resulting from step b. to the value of 10, d. mixing the solutions from step c. in a reactor to obtain a suspension, e. centrifuging the suspension from step d. to obtain precipitate, f. purifying the precipitate from step e. by rinsing it four times with ultrapure water and centrifuging, g. drying the precipitate from step f. at 50° C. for 12-24 h, h. grinding the precipitate from step g. in a ball mill for 10 minutes at a speed of 150 rpm, wherein step d. is carried out in a continuous or batch reactor.

The present invention relates to a formulation of nanoparticles of calcium phosphate, including hydroxyapatite, said nanoparticles being modified with lecithin, including phosphatidylcholine, said formulation having an enhanced cellular uptake and being a carrier for drugs from the group of bisphosphonates, e.g., sodium alendronate. The invention also relates to a method of preparing such a formulation.

A method of preparing lecithin-modified hydroxyapatite nanoparticles is described in patent 229015 and patent application P.434278. The former of the documents discloses a method of batch preparation of hydroxyapatite nanoparticles in the presence of lecithin, with the lecithin having two functions: it is a means of controlling the size and shape of the particles and a means of enhancing their biocompatibility. The patent indirectly demonstrates the effect of lecithin modification on biocompatibility properties. The latter of the documents discloses a reactor for the continuous synthesis of lecithin-modified hydroxyapatite nanoparticles. Hydroxyapatite nanoparticles can be used in therapies because they are morphologically and chemically similar to the mineral part of the bone. Such uses relate to the regeneration of bone injuries and defects and the promotion of the rebuilding and remodelling of the mineral part of bone tissue.

Bisphosphonates are a group of chemical compounds in which two hydrolysis-resistant —C—P(O)—(OH)₂ groups are present. These compounds exhibit strong affinity to the mineral bone components, being apatites (Zhang, S., Gangal, G. and Uludaǧ, H. Chem Soc Rev 36, 507-531 (2006)). They are also active in regulating bone remodelling processes, which is why they have been essential in treating osteoporosis and bone cancers for a few decades. The group of bisphosphonates includes alendronic acid or, more typically, its salt, sodium alendronate, but its bioavailability when administered orally is very low (below 1%) (Porras, A. G., Holland, S. D. & Gertz, B. J. Clin Pharmacokinet 36, 315-328 (1999)). Moreover, when administered orally, the drug generates several side effects in the upper gastrointestinal tract, the most serious of which is a higher risk of cancer onset (Sun, K., Liu, J. M., Sun, H. X., Lu, N. & Ning, G. Osteoporosis Int 24, 279-286 (2013)). Therefore, it is necessary to propose suitable pharmaceutical bisphosphonate formulations for oral administration, improving the drug's bioavailability and reducing its side effects.

The preparation of formulations comprising hydroxyapatite nanoparticles and alendronate is known in the art because of their potential dual therapeutic effect.

J. Neamtu et al., J Therm Anal Calorim, 2017, 127:1567-1582E show a method of synthesis of alendronate-hydroxyapatite nanoparticles from calcium nitrate, diammonium hydrogen phosphate and alendronate by chemical precipitation for use in biomaterials. The formulation of nanoparticles which comprise alendronate was obtained in a batch reactor in two ways: 1) addition of alendronate solution to the mixture when hydroxyapatite nanoparticles have precipitated, 2) addition of alendronate solution to one of the reagents. The publication also describes employing crystallographic, spectroscopic and thermal methods to chemically and physically characterise composites and determine the alendronate's release profile. No additional modification was indicated in the study that would potentially improve the bioavailability of the resulting formulation.

E. Boanini et al., Biomaterials, 29, 2008, 790-796 describe a method for preparing hydroxyapatite nanoparticles with alendronate and their in vitro interaction with osteoclasts and osteoblast-like cells. According to the tests conducted, the presence of bisphosphonates in nanocrystals inhibits the proliferation of osteoclasts. It promotes the growth and differentiation of osteoblasts, particularly those cultured in the presence of nanoparticles with relatively high alendronate content, which exhibit elevated values of alkaline phosphatase and type I collagen activity. Again, the authors did not describe additional modifications to the particles to improve the formulation's bioavailability potentially.

R. Bosco et al., Applied Surface Science 328, 2015, 516-524 shows a method of coating titanium bone implants with alendronate-hydroxyapatite nanocrystals with alendronate content of about 30 wt %. The coating was achieved by electrostatic spray deposition (ESD) or immersion in a dissolved bisphosphonate solution. The described results of in vitro tests demonstrated an active role of nHA_{ALE} crystals in reducing the number of viable osteoclasts.

Numerous patent publications also disclose the use of a composition comprising hydroxyapatite and alendronate. CN108478872A describes a method of manufacturing bone cement of PMMA, additionally comprising a hydroxyapatite-alendronate nanocomposite for use in filling bone defects. Patent publication WO2009035265 presents a method of synthesising and using calcium phosphates as systems based on microparticles for oral administration of drugs from the group of bisphosphonates, including alendronate, in the treatment of osteoporosis. The disclosed drug content of the formulation is in the range of 1-50 wt %, based on 100 wt % hydroxyapatite, and the synthesis is carried out by crystallisation from a water-oil emulsion system.

U.S. Pat. No. 8,158,153 discloses a formulation for oral administration based on nanoparticles (with their size not exceeding 2000 nm) with an active bisphosphonate cation, said formulation comprising a penetration enhancing agent and a chelating agent. The disclosed formulation does not comprise calcium phosphates, including hydroxyapatite. U.S. Pat. No. 6,783,772B1 discloses an oral composition in the form of a tablet comprising therapeutic amounts of sodium alendronate for releasing sodium alendronate in the stomach and through the oesophagus. The formulation comprises a compressed granulated core with sodium alendronate embedded in a therapeutically inert sugar-based fibrous matrix. EP 2 548 441 B1 discloses a sustained-release formulation comprising bisphosphonate for intravenous administration.

Although there are various types of formulations in the literature to ensure adequate delivery of bisphosphonates, including alendronate as an active agent in osteoporosis therapy, there is still a need for formulations with improved cellular uptake and increased therapeutic effect.

The subject matter of the invention is a formulation of nanoparticles of calcium phosphate, preferably hydroxyapatite, said nanoparticles being modified with lecithin, preferably phosphatidylcholine, said formulation having an enhanced cellular uptake and being a carrier for bisphosphonate, characterised in that the bisphosphonate is selected from the group of bisphosphonate drugs approved for medical use, the group comprising alendronate and zoledronate, with the bisphosphonate encapsulated in calcium phosphate nanoparticles in an amount up to 40% by mass and nanoparticles being less than 200 nm in size.

Preferably, alendronate is introduced to the formulation as sodium alendronate at a concentration in the range of 5 mM-15 mM based on the volume of the reaction mixture.

Preferably, zoledronate is introduced to the formulation as zoledronic acid at a concentration of 5 mM based on the volume of the reaction mixture.

The subject matter of the invention is also a method of obtaining calcium phosphate nanoparticles comprising the steps of: a) dissolving Ca(NO₃)₂·4H₂O in a lecithin solution, b) dissolving (NH₄)₂HPO₄ in a bisphosphonate solution, adjusting the pH of the solution resulting from step a) and of the solution resulting from step b) to the value of 10, mixing the resulting solutions in a reactor to obtain a suspension, then centrifuging the suspension to obtain precipitate, purifying the precipitate by rinsing it four times with ultrapure water and centrifuging and drying the precipitate at 50° C. for 12-24 h, grinding the precipitate in a ball mill for 10 minutes at a speed of 150 rpm, wherein the mixing of the solutions in the reactor to obtain the suspension is carried out in a continuous or batch reactor.

Preferably, the reactor used in the method is a continuous reactor.

### BRIEF DESCRIPTION OF THE FIGURES

FIG. 1. shows scanning electron microscope images of particles comprising lecithin and alendronate, said particles obtained in the method with the concentration of alendronate used to precipitate being a) 5 mM, c) 10 mM, e) 15 mM based on the volume of the reaction mixture and g) of particles comprising lecithin only, and images of particles comprising alendronate, said particles obtained in the method with the concentration of alendronate used to precipitate being b) 5 mM, d) 10 mM, f) 15 mM based on the volume of the reaction mixture and h) of particles of pure hydroxyapatite.

FIG. 2. shows confocal laser scanning microscopy images made by superimposing a transmitted light image over a fluorescence image of the hydroxyapatite nanoparticles excited with a laser with a wavelength of 488 nm; the images illustrating bone cells in contact with a) pure hydroxyapatite, b) lecithin-modified hydroxyapatite. The arrows indicate particles taken up by the cells. The scale is 25 km.

FIG. 3. shows the results of alendronate release (mean mass of released alendronate (mg) in relation to the mass of formulation sample used for the test (0.5 g)) from hydroxyapatite particles comprising alendronate and lecithin and particles comprising alendronate only a) in a release medium having pH=7.4 (n=3), b) in a release medium having pH=5.0 (n=3).

Preferred embodiments of the invention include, above all, formulations of hydroxyapatite nanoparticles modified with lecithin, said formulations comprising bisphosphonate, preferably alendronic acid or its sodium salt. Such preferred formulations are obtained in a precipitation reaction in a flow reactor, where up to about 500 mg sodium alendronate is used for the reaction, which is the maximum allowed content of sodium alendronate (water solubility: 10 mg/mL) in the reagent solution (50 mL) in a precipitation reaction. Additionally, such formulations are precipitated in the presence of lecithin (about 98% phosphatidylcholine) to enhance cellular uptake of the formulation nanoparticles and, thus, potentially enhance the formulation's bioavailability.

In the following, the invention is described in detailed embodiments. However, the examples provided are not limiting.

### Example 1—nHAp-LE-AL 5 mM

0.3 g of lecithin (phosphatidylcholine, Lipoid S PC-3, Lipoid GmbH, Germany) were weighed and dissolved in 50 mL ultrapure water at about 60° C. for about 30 minutes with stirring. Then 5.904 g of calcium (V) nitrate tetrahydrate —Ca(NO₃)₂·4H₂O were weighed and dissolved in a previously cooled lecithin solution. 162.5 mg sodium alendronate (5 mM based on 100 mL of the reaction mixture) was weighed and dissolved in 50 mL ultrapure water. Then 1.981 g di-ammonium hydrogen phosphate —(NH₄)₂HPO₄ was weighed and dissolved in sodium alendronate solution. Using ammonia water, the pH of both reagents was set to 10. The prepared solutions were mixed in a flow reactor at room temperature at a constant dosing rate of 500 mL/h. A Y geometry flow reactor was used with inlet channels of 50 mm length and an outlet channel of 10 mm length and dimensions of square cross-sections of channels of 1 mm×1 mm. The suspension (100 mL) obtained in the receiving tank was centrifuged for 30 minutes at a speed of 4500 rpm. The supernatant was then decanted, and the residual precipitate was purified four times by rinsing with ultrapure water and centrifuging (10 minutes, 4500 rpm). The final product was allowed to dry at 50° C. for about 12 h and then ground in a ball mill for 10 minutes at 150 rpm. The obtained product was in the form of a powder of an off-white colour. The product was designated with the acronym nHAp-LE-AL 5 mM.

### Example 2—nHAp-LE-AL 10 mM

0.3 g of lecithin (phosphatidylcholine, Lipoid S PC-3, Lipoid GmbH, Germany) were weighed and dissolved in 50 mL ultrapure water at about 60° C. for about 30 minutes with stirring. Then 5.904 g of calcium (V) nitrate tetrahydrate —Ca(NO₃)₂·4H₂O were weighed and dissolved in a previously cooled lecithin solution. 325.0 mg sodium alendronate (10 mM based on 100 mL of the reaction mixture) was weighed and dissolved in 50 mL ultrapure water. Then 1.981 g di-ammonium hydrogen phosphate —(NH₄)₂HPO₄ was weighed and dissolved in sodium alendronate solution. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp-LE-AL 10 mM.

### Example 3—nHAp-LE-AL 15 mM

0.3 g of lecithin (phosphatidylcholine, Lipoid S PC-3, Lipoid GmbH, Germany) were weighed and dissolved in 50 mL ultrapure water at about 60° C. for about 30 minutes with stirring. Then 5.904 g of calcium (V) nitrate tetrahydrate —Ca(NO₃)₂·4H₂O were weighed and dissolved in a previously cooled lecithin solution. 487.5 mg sodium alendronate (15 mM based on 100 mL of the reaction mixture) was weighed and dissolved in 50 mL ultrapure water. Then 1.981 g di-ammonium hydrogen phosphate —(NH₄)₂HPO₄ was weighed and dissolved in sodium alendronate solution. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp-LE-AL 15 mM.

### Example 4—nHAp-LE-ZL 5 mM

0.3 g of lecithin (phosphatidylcholine, Lipoid S PC-3, Lipoid GmbH, Germany) were weighed and dissolved in 50 mL ultrapure water at about 60° C. for about 30 minutes with stirring. Then 5.904 g of calcium (V) nitrate tetrahydrate —Ca(NO₃)₂·4H₂O were weighed and dissolved in a previously cooled lecithin solution. 145.1 mg zoledronic acid 1-hydrate (5 mM based on 100 mL of the reaction mixture) was weighed and dissolved in 50 mL ultrapure water. Then 1.981 g di-ammonium hydrogen phosphate —(NH₄)₂HPO₄ was weighed and dissolved in a zoledronate solution. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp-LE-ZL 5 mM.

### Example 5 (Comparative)—nHAp-AL 5 mM

5.904 g of calcium (V) nitrate tetrahydrate —Ca(NO₃)₂·4H₂O were weighed and dissolved in 50 mL of ultrapure water. 162.5 mg sodium alendronate (5 mM based on 100 mL of the reaction mixture) was weighed and dissolved in 50 mL ultra-pure water. Then 1.981 g di-ammonium hydrogen phosphate —(NH₄)₂HPO₄ was weighed and dissolved in sodium alendronate solution. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp-AL 5 mM.

### Example 6 (comparative)—nHAp-AL 10 mM

5.904 g of calcium (V) nitrate tetrahydrate —Ca(NO₃)₂·4H₂O were weighed and dissolved in 50 mL of ultrapure water. 325.0 mg sodium alendronate (10 mM based on 100 mL of the reaction mixture) was weighed and dissolved in 50 mL ultra-pure water. Then 1.981 g di-ammonium hydrogen phosphate —(NH₄)₂HPO₄ was weighed and dissolved in sodium alendronate solution. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp-AL 10 mM.

### Example 7 (Comparative)—nHAp-AL 15 mM

5.904 g of calcium (V) nitrate tetrahydrate —Ca(NO₃)₂·4H₂O were weighed and dissolved in 50 mL of ultrapure water. 487.5 mg sodium alendronate (15 mM based on 100 mL of the reaction mixture) was weighed and dissolved in 50 mL ultra-pure water. Then 1.981 g di-ammonium hydrogen phosphate —(NH₄)₂HPO₄ was weighed and dissolved in sodium alendronate solution. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp-AL 15 mM.

### Example 8 (Comparative)—nHAp

5.904 g of calcium (V) nitrate tetrahydrate —Ca(NO₃)₂·4H₂O were weighed and dissolved in 50 mL of ultrapure water. Then 1.981 g di-ammonium hydrogen phosphate —(NH₄)₂HPO₄ was weighed and dissolved in 50 mL of ultrapure water. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp.

### Example 9 (Comparative)—nHAp-LE

0.3 g of lecithin (phosphatidylcholine, Lipoid S PC-3, Lipoid GmbH, Germany) were weighed and dissolved in 50 mL ultrapure water at about 60° C. for about 30 minutes with stirring. Then 5.904 g of calcium (V) nitrate tetrahydrate —Ca(NO₃)₂·4H₂O were weighed and dissolved in a previously cooled lecithin solution. Then 1.981 g di-ammonium hydrogen phosphate —(NH₄)₂HPO₄ was weighed and dissolved in 50 mL of ultrapure water. Using ammonia water, the pH of both reagents was set to 10. The prepared solutions were mixed in a flow reactor under the conditions described in Example 1. The suspension (100 mL) obtained in the receiving tank was purified, and the final product was prepared as in Example 1. The obtained product was in the form of a powder of an off-white colour. The product was designated with the acronym nHAp-LE.

### Example 10—Scanning Electron Microscopy

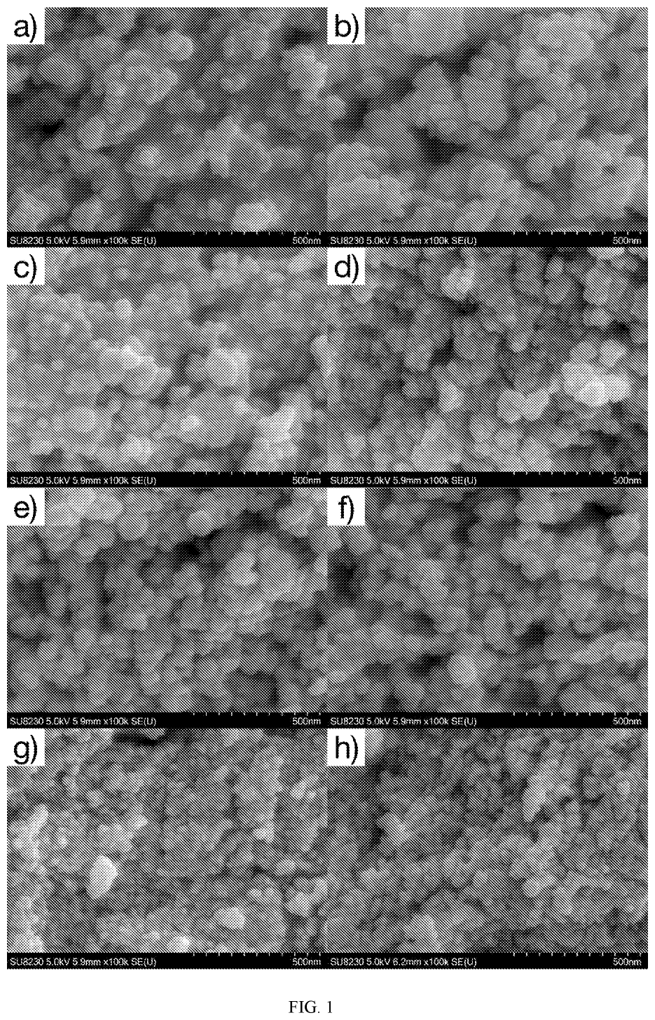

The size of the individual hydroxyapatite particles was determined from scanning electron microscope (SEM) images. Before imaging, the hydroxyapatite samples were sputtered with a 10 nm gold-palladium conductive layer. The Q150T (Quorum, UK) sputter coater was used, while the SEM images were taken with a scanning electron microscope with the SU8230 (Hitachi, Japan). The photographs of the test particles are collected in FIG. 1. The particles with alendronate added to synthesis showed a significant increase in the size of a single particle compared to those not comprising this active substance.

### Example 11—Measurement of Particle Sizes

The SEM images were digitally analysed to determine the particle size of the hydroxyapatite. The analysis was performed with the ImageJ program (version 2.3.0/1.53f). For each sample tested, 100 independent particle size measurements were made. The result as mean with standard deviation is given in Table 1. A significant increase in particle size was observed when sodium alendronate was added to the synthesis.

### Example 12—Measurement of Particle Size in Aqueous Suspension

Particle size measurements in aqueous suspension were made using Zetasizer Nano ZS (Malvern Instruments Ltd., UK) apparatus equipped with a red laser with a wavelength of 633 nm. The samples for analysis were prepared by making a 1% (w/v) suspension of hydroxyapatite powder in 10 mM KNO₃. The samples were additionally exposed to ultrasonics for about 10 minutes using ultrasonic homogeniser UP100H (Hielscher Ultrasonics, Germany) and then diluted 50-fold and filtered using a syringe filter with a pore size of 0.45 m to remove contaminants and dust. Measurements were made at a constant temperature of 25° C. in five replicates. The result is given in Table 1 as a mean with standard deviation. Means were calculated from the number-weighted particle size distributions. Compared to particles not comprising the active substance, the increase in the mean size of the particles comprising sodium alendronate indicates the possible encapsulation of the active agent inside the particles and/or its attachment to the particle surface.

### Example 13—Measurement of Zeta Potential

Zeta potential measurements were made using Zetasizer Nano ZS (Malvern Instruments Ltd., UK) apparatus equipped with a red laser with a wavelength of 633 nm. The samples for analysis were prepared by making a 1% (w/v) suspension of hydroxyapatite powder in 10 mM KNO₃. The samples were additionally exposed to ultrasonics for about 10 minutes using ultrasonic homogeniser UP100H (Hielscher Ultrasonics, Germany) and then diluted 50-fold. Measurements were made at a constant temperature of 25° C. in five replicates. The result is given as a mean with standard deviation in Table 1. Changes in the zeta potential of the surfaces of the tested particles are insignificant, and the measured values indicate that the obtained hydroxyapatite powders comprising lecithin and sodium alendronate and powders comprising sodium alendronate only tend to form agglomerates in an aqueous environment. However, these agglomerates can be effectively dispersed by methods known in the art.

### Example 14—Thermogravimetric Analysis

Thermogravimetric analysis (TGA) measurements were made using Mettler Toledo TGA/DSC 3+ apparatus in a temperature range of 30 to 1000° C. at a heating rate of 10° C./min. The tests were conducted at a continuous air flow rate of 30 mL/min. Based on the results obtained, the content of sodium alendronate in the prepared formulations was determined using the correlations:
for hydroxyapatite not modified with lecithin:

$$AL ( \% by mass ) = \frac{Δ {w}_{nHAp - AL} - Δ {w}_{nHAp}}{Δ {w}_{AL} - Δ {w}_{nHAp}} \times 100 \%$$

for hydroxyapatite modified with lecithin:

$$AL ( \% by mass ) = \frac{Δ {w}_{nHAp - LE - AL} - Δ {w}_{LE} LE - Δ {w}_{nHAp} ( 1 - LE )}{Δ {w}_{AL} - Δ {w}_{nHAp}} \times 100 \%$$

where:
Δw_{AL}, Δw_{LE}, Δw_{nHAp}, Δw_{nHAp-AL}, Δw_{nHAp-LE-AL} are the mass loss of the samples of sodium alendronate (AL), lecithin (LE), hydroxyapatite nanoparticles (nHAp), hydroxyapatite nanoparticles comprising alendronate (nHAp-AL) and hydroxyapatite nanoparticles comprising lecithin and alendronate (nHAp-LE-AL), respectively, in the TGA test, expressed in mass fraction;
LE—is the lecithin content of the hydroxyapatite nanoparticles powder expressed in mass fraction.

The calculated sodium alendronate contents (% by mass) of the formulations powders of the examples according to the invention (Examples 1-4) and the comparative examples (Examples 5-9) are summarised in Table 1.

TABLE 1

| | | Mean | | |
|---|---|---|---|---|
| | | particle | Zeta | |
| | | size in | potential | AL content |
| | Particle | aqueous | in aqueous | in powders |
| | size ± SD | suspension ± | suspension ± | (TGA) |
| Formulation | (SEM) [nm] | SD [nm] | SD [mV] | [% by mass] |
| Formulation of | 53 ± 14 | 49 ± 6 | −6.7 ± 0.5 | 34.8 |
| Example 1 | | | | |
| Formulation of | 55 ± 15 | 41 ± 9 | −9.3 ± 0.6 | 38.5 |
| Example 2 | | | | |
| Formulation of | 64 ± 15 | 60 ± 10 | −5.2 ± 1.5 | 39.3 |
| Example 3 | | | | |
| Formulation of | 40 ± 9 | 79 ± 29 | 0.0 ± 0.1 | 22.5 |
| Example 4 | | | | |
| Formulation of | 43 ± 13 | 43 ± 12 | −7.0 ± 1.3 | 27.0 |
| Example 5 | | | | |
| Formulation of | 51 ± 15 | 54 ± 25 | −13.1 ± 2.4 | 25.8 |
| Example 6 | | | | |
| Formulation of | 56 ± 15 | 48 ± 21 | −11.6 ± 1.3 | 26.5 |
| Example 7 | | | | |
| Formulation of | 22 ± 5 | 30 ± 7 | −11.6 ± 1.8 | — |
| Example 8 | | | | |
| Formulation of | 21 ± 5 | 36 ± 6 | −7.8 ± 1.7 | — |
| Example 9 | | | | |

### Example 15—Measurement of Enhanced Cellular Uptake of Hydroxyapatite Nanoparticles

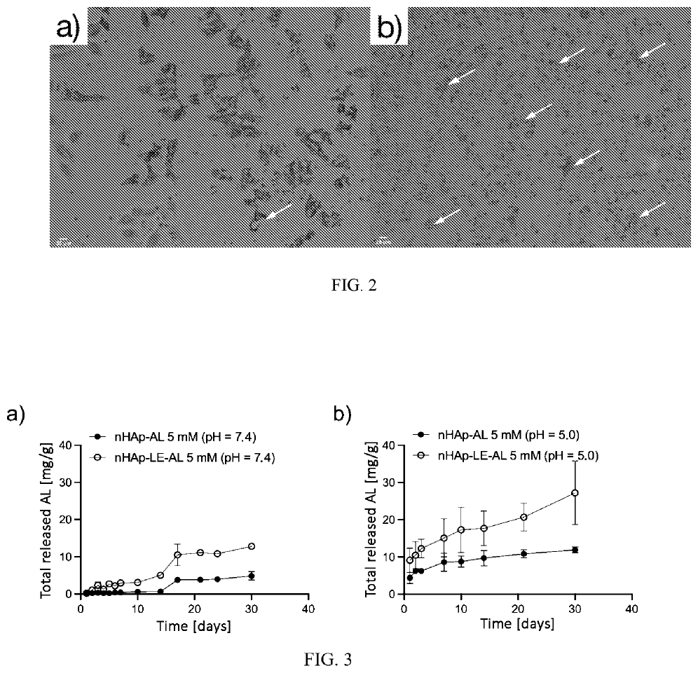

To test the enhanced cellular uptake of hydroxyapatite nanoparticles, which are surface-modified with lecithin, human bone sarcoma cells of the MG63 line (Sigma-Aldrich, Germany) were used. Before the experiment, cells were grown in DMEM (Dulbecco's Modified Eagle Medium) culture medium supplemented with 10% v/v bovine serum and antibiotics (100 U/mL penicillin, 100 mg/mL streptomycin). The cell suspension having a concentration of 10⁵ cells/mL, was transferred to the surface of sterile round cover glasses placed in the wells of a 24-well plate. 1 mL of cell suspension was introduced to each well. Two replicates were prepared for each hydroxyapatite powder tested (n=2). Lecithin-modified hydroxyapatite (nHAp-LE) and pure hydroxyapatite (nHAp) powder samples were prepared for the test. The powder samples for the test were prepared by making a 1% (w/v) suspension of hydroxyapatite powder in phosphate buffer (PBS, pH=7.4). The samples were additionally exposed to ultrasonics for about 10 minutes using ultrasonic homogeniser UP100H (Hielscher Ultrasonics, Germany) and then diluted 50-fold and filtered using a syringe filter with a pore size of 0.45 m to remove contaminants and dust and to sterilise the samples additionally. After 4 h incubation of osteoblast cells with nHAp-LE and nHAp nanoparticle suspensions, the suspensions were drained from the cells, and the cells were rinsed twice with sterile PBS. The cells were fixed by a method known in the art, using paraformaldehyde. The cover glasses, on which the cells were cultured, were transferred to wetted microscope slides and observed using Zeiss LSM880 (Zeiss, Germany) confocal laser scanning microscope. Photographs of preparations were taken using transmitted light and fluorescence of hydroxyapatite nanoparticles excited with a laser with a wavelength of 488 nm. In this manner, the uptake of nHAp-LE and nHAp nanoparticles by bone cells is shown (FIG. 2). The lecithin-modified nanoparticles were more readily taken up by bone cells in the test presented here.

### Example 16—Measuring the Rate of Alendronate Release from the Formulation

500 mg of nHAp-LE-AL 5 mM powder (174.0 mg AL powder) and nHAp-AL 5 mM powder (135.2 mg AL powder) were weighed, and each was suspended in 10 mL of the release medium. This way, 3 samples of each tested material per experiment (n=3) were prepared. PBS with a pH of 7.4 (Experiment 1) and 5.0 (Experiment 2) was used as the release medium. Experiments testing the alendronate release from the formulation of hydroxyapatite nanoparticles according to the invention were conducted for 30 days. During the experiments, the temperature of the release test vessels was maintained at 37° C. and the suspensions were stirred. Samples were taken at set intervals. Before sampling, the test suspensions were centrifuged (10 minutes, 4500 rpm). After centrifugation, two 0.5 mL samples of supernatant were taken from each release rate test vessel and the vessel was immediately replenished with the release medium in the volume taken. Each sample was then mixed with 0.5 mL of a 0.4 mM solution of FeCl₃ in 4 M HClO₄. The absorbance of the thus prepared mixture (at a wavelength of 290 nm) was measured, and the concentration of alendronate was determined based on a previously prepared standard curve. The results are presented as the mean mass of alendronate released (mg) in relation to the mass of the formulation sample used for the test (0.5 g)±SD (n=3). The release of the active substance from the formulation was faster under conditions with the lower pH tested, which corresponds to the pH in the endosomes of the cells.

1. Formulation of nanoparticles of calcium phosphate, preferably hydroxyapatite, said nanoparticles being modified with lecithin, preferably phosphatidylcholine, said formulation having an enhanced cellular uptake and being a carrier for bisphosphonate, characterised in that
bisphosphonate is selected from the group of bisphosphonate drugs approved for medical use, the group comprising alendronate and zoledronate,
bisphosphonate is encapsulated in calcium phosphate nanoparticles in an amount up to 40% by mass,
and the nanoparticles are less than 200 nm in size.

2. The formulation of claim 1, characterised in that alendronate is introduced to the formulation as sodium alendronate at a concentration in the range 5 mM-15 mM based on the volume of the reaction mixture.

3. The formulation of claim 1, characterised in that zoledronate is introduced to the formulation as zoledronic acid at a concentration of 5 mM based on the volume of the reaction mixture.

4. Method of obtaining the formulation of claim 1 comprising the steps:
a. dissolving Ca(NO₃)₂·4H₂O in a lecithin solution,
b. dissolving (NH₄)₂HPO₄ in a bisphosphonate solution,
c. adjusting the pH of the solution resulting from step a. and of the solution resulting from step b. to the value of 10,
d. mixing the solutions from step c. in a reactor to obtain a suspension,
e. centrifuging the suspension from step d. to obtain precipitate,
f. purifying the precipitate from step e. by rinsing it four times with ultrapure water and centrifuging,
g. drying the precipitate from step f. at 50° C. for 12-24 h,
h. grinding the precipitate from step g. in a ball mill for 10 minutes at a speed of 150 rpm,
wherein step d. is carried out in a continuous or batch reactor.

5. The method of claim 4, characterised in that the reactor is a continuous reactor.
